(19) United States
(12) Patent Application Publication
(10) Pub. No.: US 2024/0200276 A1
(43) Pub. Date: Jun. 20, 2024

(54) DEVICE AND METHOD FOR SIMULTANEOUSLY PRODUCING A PLURALITY OF CONTAINERS COMPRISING FIBERS

(71) Applicant: Krones AG, Neutraubling (DE)

(72) Inventors: Frank WINZINGER, Regensburg (DE); Thomas ALBRECHT, Beilngries (DE); Daniel PETER, Hemau (DE); Andreas MEYER, Nittenau (DE); Eduard HANDSCHUH, Donaustauf (DE); Andreas HIPPEL, Wörth (DE)

(21) Appl. No.: 18/545,622

(22) Filed: Dec. 19, 2023

(30) Foreign Application Priority Data
Dec. 20, 2022 (DE) 102022134094.5

(51) Int. Cl. D21J 3/10; A61L 2/10; A61L 2/26; B65B 3/02; B65B 7/16; B65B 61/26; D21C 9/08; D21F 1/66; D21F 13/00; D21G 9/00

(52) U.S. Cl. CPC D21J 3/10; A61L 2/10; A61L 2/26; B65B 3/02; B65B 7/16; B65B 61/26; D21C 9/08; D21F 1/66; D21F 13/00; D21G 9/0018; D21G 9/0027

(57) ABSTRACT

The disclosure relates to a device for simultaneously producing a plurality of containers comprising fibers, using fiber-containing pulp. The disclosure furthermore relates to a method for simultaneously producing a plurality of containers comprising fibers, using fiber-containing pulp by means of the device.

### CROSS REFERENCE TO RELATED APPLICATION

The present application claims priority to German Patent Application No. 10 2022 134 094.5 filed on Dec. 20, 2022. The entire contents of the above-listed application are hereby incorporated by reference for all purposes.

### TECHNICAL FIELD

The disclosure relates to a device and a method for simultaneously producing a plurality of containers comprising fibers.

### BACKGROUND

It is usual to produce containers comprising fibers, for example using the pulp comprising the fibers, in porous molds. The pulp is introduced into the mold and, by inflating an elastic balloon which is introduced into the mold and presses the pulp against the mold, a container comprising fibers can be formed. With this method of production, long cycle times can result.

### SUMMARY

### Object

The object of the disclosure is to provide a device and a method for simultaneously producing a plurality of containers comprising fibers, which enable a continuous, simultaneous and finished forming of a plurality of containers comprising fibers.

### Achievement

The object is achieved by the device and the method for simultaneously producing a plurality of containers comprising fibers, as described herein.

The device according to the disclosure for the simultaneous production of a plurality of containers comprising fibers, using fiber-containing pulp.

Simultaneous production can mean a temporally simultaneous production by means of the device.

The fibers may comprise lignin, banana leaves and/or wild quinine. The fibers may, for example, comprise cellulose fibers, fibers of conifers, of leafy woody plants and/or plane trees and/or grasses, reeds and/or bamboo or the like. The fibers may comprise silk threads, spider silk, algae, natural fibers (such as Danube silphie fibers, hemp, maize, cotton), banana peel, orange peel, grass, straw, potato starch or processed cow dung. It is also possible to provide cellulose fibers which originate from a process by which they were artificially grown. In the event of material bottlenecks of wood as the basic material for a fluid mass with fibers, these alternative materials can completely or partially replace the basic material.

The fibers can comprise fiber mixtures made of non-wood material, for example cotton, hemp and/or textile fibers.

For example, the fibers may comprise viscose fibers. A viscose fiber is an artificial fiber made of regenerated cellulose, wherein the viscose fibers have, as starting base, 100% cellulose treated in a multi-stage process. The viscose fibers may comprise a flat or cable-shaped structure, a trilobal shape or a double trilobal structure. The fibers may comprise a flat or hollow structure or a corrugated or rough outer surface.

Various types of fibers may also be used in combination. For example, fibers with different outer surfaces and/or shapes and/or hollow structure and/or flat structure and/or of different materials may be combined.

Due to the natural origin of the fibers, they may be biodegradable. In addition, they are sustainable and renewable.

The fibers can be used in a pulp which can be used, for example, to produce the container comprising fibers. The pulp may be or comprise a mixture of water, for example with additives, and fibers. The pulp may comprise a mixture of fibers and liquid components.

The containers may be or comprise bottles, cans or cups.

The device may comprise a plurality of stations for the respective forming of a container comprising fibers. The plurality of stations may be arranged on a rotary machine.

Each station may comprise a preform mold and a press mold, wherein the preform mold may, for example, comprise a lattice structure. The preform mold and/or the press mold may each comprise a negative shape of an outer contour of the container to be produced. The preform mold may be used for preforming the container to be produced. In the press mold, a container preformed in the preform mold can be pressed, for example in order to press out at least a part of the water of the pulp and/or to press the fibers of the pulp into one another.

The device may comprise two sub-devices, wherein the first sub-device may comprise preform molds and the second sub-device may comprise press molds. The two sub-devices may each be designed as rotary devices with a plurality of stations.

For example, the device and/or one or both sub-devices may comprise a plurality of stations with molds, wherein, for example, the stations can be arranged at equidistant intervals along the circumference of a wheel (rotary device) which rotates in a clocked or continuous manner.

The device may furthermore comprise a device for feeding fiber-containing pulp. The device may, for example, comprise a rotary distributor. For example, the rotary distributor may conduct pulp and/or fibers from a stationary part of the device to the stations in the rotating part. In the case of two sub-devices, a rotary distributor for pulp and/or fibers may be present only in the first sub-device.

A plurality of rotary distributors or rotary distribution tracks may also be provided (e.g., concentrically in the same rotary distributor), wherein compressed air can be transported to the stations in a further track or a further rotary distributor, in addition to the pulp and/or the fibers. In the case of two sub-devices, the first and/or the second sub-device may comprise a compressed air rotary distributor.

A compressed air rotary distributor can be connected to a compressor on its stationary side. The device may comprise the compressor or the compressor may be provided independently of the device.

Piping of the rotary distributor may be designed in such a way that the fiber-containing pulp can pass through the pipes, for example without clogging them.

The device may furthermore comprise a device for collecting and returning at least a part of the water comprised in the fiber-containing pulp. The part of the water can be removed from the pulp in the preform mold and/or in the press mold, for example by pressing and/or by centrifugal forces acting on the container to be produced.

The device may furthermore comprise at least one transmission device for applying pressing force to at least two of the press molds. In this way, only one transmission device may be required for a plurality of press molds and not, for example, one assigned transmission device for each press mold.

The at least one transmission device may comprise a motor and at least one lever, for example at least one toggle lever which can be driven by the motor, whereby a compressive pressure can be exerted on the at least two of the press molds. By means of the lever, a pressing force can be exerted on the press mold from the outside.

The at least one transmission device may comprise a supply device for compressed air, wherein the supply device can be connected to at least two elastic balloons (sometimes also called bladders) by means of a piping system for distributing the compressed air, wherein each of the elastic balloons can be provided for introduction into one of the press molds and for inflation by means of the compressed air.

The transmission device may comprise the at least two elastic balloons. By means of the balloons, a pressing force can be exerted on the press mold from the inside.

After pressing, the compressed air of a balloon of a station can be reused at least partially to build up pressure in the balloon of another station. For this purpose, the stations can be connected to one another by lines.

The device may be designed to provide a separable connection of the plurality of containers using the fiber-containing pulp during the simultaneous production of the plurality of containers comprising fibers. For example, the separable connection may comprise one or more perforations. For example, the separable connection may comprise bridges and/or a continuous planar connection between the plurality of containers.

For example, a six-pack of containers may be provided, which can be arranged, for example, in a matrix of 2×6 or 2×3. By means of the separable connection, one of the containers can be separated from the remaining containers, wherein the connection between the remaining containers may be maintained.

The device may furthermore comprise a removal device for removing the containers comprising fibers from the preform molds or press molds. For example, the device may furthermore comprise a filling device for simultaneously filling the plurality of containers with product (e.g., a fluid product, such as a beverage or the like). For example, the device may furthermore comprise a closing device for simultaneously closing the plurality of containers. For example, the device may furthermore comprise a labeling device for simultaneously or successively labeling the containers.

The device may comprise a rotary distributor for feeding fiber-containing pulp. Piping of the rotary distributor may be designed in such a way that the fiber-containing pulp can pass through the pipes, for example without clogging them.

The device for collecting and returning at least a part of the water which may be comprised in the fiber-containing pulp can be arranged below the plurality of stations in the direction of action of gravity. The at least one part of the water can then drip and/or flow under the action of gravity into the device for collecting and returning. For example, the device for collecting and returning may comprise a trough, for example an annular trough, for example taking the form of a water lock. For example, a lateral surface of a circular cone inclined toward the trough may also be provided below the plurality of stations in the direction of action of gravity. The at least one part of the water can then flow along the lateral surface and reach the trough under the action of gravity.

The device may comprise a separate rotary distributor track or a separate rotary distributor for returning pressed-out medium. The pressed-out medium may comprise water and/or fractions of fibers and/or other components of the pulp, which can be pressed out, for example, during pressing of the pulp or during molding of the container comprising fibers and/or can thereby come loose.

Alternatively, a drip channel, which can serve as a “rotary distributor” and in which liquid and/or the pressed-out medium can be collected and returned, may be provided below the stations.

A rotary distributor for returning pressed-out medium, e.g., a separate rotary distributor track or a separate rotary distributor, can be connected to a mixing container on its stationary side.

The device for collecting and returning at least a part of the water comprised in the fiber-containing pulp may comprise the rotary distributor.

The device may furthermore comprise a device for producing the fiber-containing pulp, wherein the device for producing the fiber-containing pulp may be line-connected by a feed line to the device for collecting and returning at least a part of the water comprised in the fiber-containing pulp, for feeding the at least one part of the water comprised in the fiber-containing pulp and for using the at least one part of the water comprised in the fiber-containing pulp for the production of further fiber-containing pulp. The pressed-out water which may correspond to the at least one part of the water comprised in the fiber-containing pulp can thus be circulated. Water consumption can be reduced. By feeding the at least one part of the water comprised in the fiber-containing pulp to the device for producing the fiber-containing pulp, it is possible to avoid conducting this water along a water cascade of the device for simultaneously producing a plurality of containers comprising fibers, using fiber-containing pulp. This water can remain directly in the device as a more or less defined volume. The device for producing the fiber-containing pulp may comprise a heating device for the fiber-containing pulp.

The feed line may comprise a disinfection device for disinfecting the at least one part of the water comprised in the fiber-containing pulp, wherein, for example, the disinfection device is designed to dispense a disinfectant into the at least one part of the water comprised in the fiber-containing pulp and/or to apply UV-A, UV-B and/or UV-C radiation to irradiate the at least one part of the water comprised in the fiber-containing pulp.

The device may furthermore comprise a regulating device for measuring a quality of the at least one part of the water comprised in the fiber-containing pulp, and for regulating the disinfection device according to a result of the measurement of the quality.

The device may comprise a rotary carousel and the stations may be provided on a circumference of the rotary carousel. The rotary carousel may be designed for an injection of the fiber-containing pulp into the preform mold and press mold. For example, an inlet valve may be assigned to each of the preform molds and press molds. For example, a heating device may be provided for the preform molds and press molds. This rotary carousel may be regarded as a first carousel if, as described below, one or two further rotary carousels can be provided. For example, the fiber-containing pulp, which can be used for the injection, may be heated. The rotary carousel mentioned here may also be referred to as the first rotary carousel in the event that more than just one rotary carousel is provided. Injection during rotation can be controlled by the inlet valves.

The injection of the fiber-containing pulp into the preform mold and press mold can take place from below upward, i.e., for example, counter to the direction of action of gravity. In the case of injection from below upward, it may be provided that the preform mold and press mold can be arranged in such a way that the produced container is then arranged upside down in the preform mold and press mold.

A rotary carousel, as well as the rotary carousels mentioned below (for differentiation, for example, denoted by “second,” “third”), may comprise a closed transport path. In addition to non-linear sections, the transport path may comprise one or more linear sections. In a plan view, the transport path may extend in an oval or circular or kidney shape.

The device may comprise a second rotary carousel which may be arranged downstream of the rotary carousel, wherein the second rotary carousel may be designed for internal drying of the fiber-containing pulp injected into the preform mold and press mold. Internal drying may take place by means of warm air.

The device may comprise a third rotary carousel, which may be arranged downstream of the rotary carousel, wherein the third rotary carousel may be designed for pressing fiber-containing pulp that has been injected into the preform mold and press mold and for applying an inner coating. For example, the third rotary carousel may be arranged in an aseptic chamber. After application of the inner coating, further drying may take place from the outside.

It may be provided that the second rotary carousel follows the rotary carousel and the third rotary carousel follows the second rotary carousel in a transport direction of the preform molds and press molds. Alternatively, it may be provided that the third rotary carousel follows the rotary carousel and the second rotary carousel follows the third rotary carousel in a transport direction of the preform molds and press molds. Alternatively, it may also be provided that only the rotary carousel and the third carousel are provided and that the second carousel is not provided in this embodiment. The designation “third” rotary carousel is retained here (even if there are only two rotary carousels in total) in order to be able to continue to unambiguously assign the features that have been assigned to this rotary carousel.

One or more transfer stars may be provided between the carousels. A transfer star may be designed as a spacing delay star.

The rotary carousel may furthermore be designed for pressing fiber-containing pulp injected into the preform mold and press mold. Pressing may take place by means of gaseous media and an overpressure.

The device may furthermore comprise a return device for returning the preform molds and press molds to the rotary carousel after the removal of containers comprising fibers. In the one embodiment, the containers comprising fibers can be removed from the preform molds and press molds at a removal position of the third rotary carousel and, in the other embodiment, can be removed from the preform molds and press molds at a removal position of the second rotary carousel. For this purpose, the preform molds and press molds can be opened. After removal, the containers comprising fibers can be fed to further elements of the device in order to be able to undergo further treatment steps there.

The device may furthermore comprise a cleaning device and an inspection device for preform molds and press molds returned by means of the return device. During a return, the preform molds and press molds may be in an open state, which they have, for example, after opening at the removal position of the third rotary carousel or of the second rotary carousel. For example, the inspection device may comprise a discharge device for defective preform molds and press molds.

The device may comprise a drying device for drying, from the outside, the containers comprising fibers and removed from the preform molds and press molds, a filling and closing device and a labeling device. For example, the drying device and the filling and closing device may be arranged in an aseptic chamber. This aseptic chamber may also comprise the third rotary carousel. For drying from the outside, the container comprising fibers may be pretensioned from the inside.

The device may be designed in such a way that the containers comprising fibers can be arrangeable in such a way that a longitudinal axis of the containers can enclose an angle of between 70° and 110° to a direction of action of gravity. The containers may be assumed to be lying.

The device may have a modular design.

The device may comprise a coating device which can be designed to coat the containers, for example with a wax, after forming. Due to the coating, the containers can be impermeable to the product located in the container. The coating device may comprise a separate rotary device with a plurality of coating stations.

For the transport of pulp, the device may comprise pumps.

In the event that the containers are pressed or formed from dry fibers, the device may comprise blowers, screw conveyors, pushers or the like for transporting the fibers.

The device may comprise a long-stator linear motor system which may comprise a plurality of movers, which can in each case transport at least one container from the pressing station to a station arranged downstream (for example, a coating station or filling station).

Furthermore, a method for simultaneously producing a plurality of containers comprising fibers, using fiber-containing pulp by means of the device, as described above or below, is provided.

By means of the device for feeding fiber-containing pulp, for example a rotary distributor, the pulp can be fed to the plurality of stations for the respective forming of a container comprising fibers. Each of the stations may comprise a preform mold and a press mold. By means of the at least one transmission device for applying pressing force to at least two of the press molds, a pressing force can be exerted on the pulp present in the stations.

A thereby pressed-out part of water, which may be comprised in the pulp, can be collected and returned by means of the device for collecting and returning, which may be arranged below the plurality of stations in the direction of action of gravity.

The pressed-out part of water (also referred to as the at least one part of the water comprised in the fiber-containing pulp) can be returned through a feed line which line-connects the device to the device for producing the fiber-containing pulp and can be used for the production of further fiber-containing pulp. The fiber-containing pulp (also the further fiber-containing pulp) can be heated by means of the heating device which can be comprised by the device for producing the fiber-containing pulp.

The feed line can comprise a disinfection device for disinfection. The at least one part of the water comprised in the fiber-containing pulp can be disinfected by means of the disinfection device.

By means of the regulating device, a quality of the at least one part of the water comprised in the fiber-containing pulp can be measured and the disinfection device can be regulated according to a result of the measurement of the quality.

Fiber-containing pulp can be injected into the preform molds and press molds by means of the rotary carousel.

By means of the second rotary carousel, an internal drying of fiber-containing pulp injected into the preform mold and press mold can take place.

By means of the third rotary carousel, a pressing of fiber-containing pulp injected into the preform mold and press mold and application of an internal coating can take place.

After removal of the containers comprising fibers, the preform molds and press molds can be returned to the rotary carousel by means of the return device.

In the one embodiment, the containers comprising fibers can be removed from the preform molds and press molds at a removal position of the third rotary carousel and, in the other embodiment, can be removed from the preform molds and press molds at a removal position of the second rotary carousel. For this purpose, the preform molds and press molds can be opened.

A cleaning can take place by means of the cleaning device, and an inspection for preform molds and press molds returned by means of the return device can take place by means of the inspection device. Defective preform molds and press molds can be discharged by means of the discharge device of the inspection device.

By means of the drying device, the containers comprising fibers and removed from the preform molds and press molds can be dried from the outside. By means of the filling and closing device, the containers comprising fibers can be filled with product and the filled containers can be closed. The filled and closed containers comprising fibers can be labeled by means of the labeling device.

For example, during the simultaneous production of the plurality of containers comprising fibers, a separable connection of the plurality of containers using the fiber-containing pulp may be provided.

The plurality of containers can be filled simultaneously with product by means of a filling device for simultaneously filling the plurality of containers. The plurality of containers filled with product can be closed simultaneously by the closing device. The plurality of closed containers can be labeled simultaneously or successively with the labeling device.

The device may furthermore comprise a central tank or a ring line which can be designed in such a way that, during injection, fiber-containing pulp can be conducted from the tank or from the ring line to the individual preform molds and press molds.

Furthermore, for example for providing fiber-containing pulp, the device may comprise a filling tank, which may be designed in such a way that a fill level in the filling tank can be kept at a given value and a process pressure can be regulated.

The preform molds and press molds may be designed to be heatable. For example, an electric heating device may be provided or a heated medium, such as oil, may be used, which can be conducted, for example, through media channels of the preform molds and press molds. By means of the heated preform molds and press molds, a drying process of injected and/or pressed-out fiber-containing pulp can be initiated in the preform molds and press molds, for example before a subsequent, separate drying process.

The use of heated fiber-containing pulp in heated preform molds and press molds or in unheated preform molds and press molds can also initiate a drying process of injected and/or pressed-out fiber-containing pulp in the preform molds and press molds, for example before a subsequent, separate drying process.

The subsequent, separate drying process can take place by means of a downstream drying device which may be comprised by the device.

### BRIEF DESCRIPTION OF FIGURES

The accompanying Figures show, by way of example, aspects and/or exemplary embodiments of the disclosure for better understanding and illustration. In the figures:

FIG. 1 shows a sectional representation of the device for simultaneously producing a plurality of containers comprising fibers, using fiber-containing pulp;

FIG. 2 shows a sectional representation of a first embodiment of a transmission device for applying pressing force to two press molds;

FIG. 3 shows a sectional representation of a second embodiment of a transmission device for applying pressing force to four press molds; and

FIG. 4 shows a plan view of a device for simultaneously producing a plurality of containers comprising fibers, using fiber-containing pulp.

### DETAILED DESCRIPTION OF FIGURES

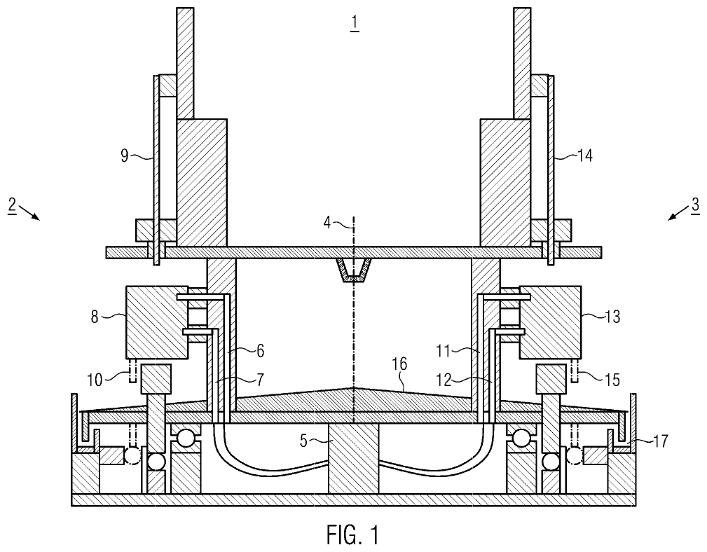

FIG. 1 shows a sectional representation of the device 1 for the simultaneous production of a plurality of containers comprising fibers, using fiber-containing pulp, wherein two stations 2, 3 for the respective forming of a container comprising fibers can be seen in the representation. The device 1 may comprise a total of two, three, four or more of the stations 2, 3. The device 1 is designed to be rotatable about an axis of rotation 4, wherein the axis of rotation is oriented perpendicularly or essentially perpendicularly to a hall floor, for example. The stations 2, 3 are arranged on a circumference of the device 1. The circumference may be the outer circumference or another circumference that can be described by the device 1.

The fiber-containing pulp can be fed simultaneously by means of a device 5 for feeding fiber-containing pulp, for example a rotary distributor, to a preform mold or press mold 8, 13 of the individual stations 2, 3. For this purpose, for example, a first pipe 6, 11 and a second pipe 7, 12 can in each case lead from the device 5 for feeding fiber-containing pulp to the preform mold or press mold 8, 13 of the respective station 2, 3. By means of the first pipe 6, 11, a different region of the preform mold or press mold 8, 13 can be supplied with the fiber-containing pulp than by means of the second pipe 7, 12. For example, fiber-containing pulp can be supplied by means of the first pipe 6, 11 to a neck/shoulder region of the container comprising fibers that is to be produced, and by means of the second pipe 7, 12 to the body/base region, for example.

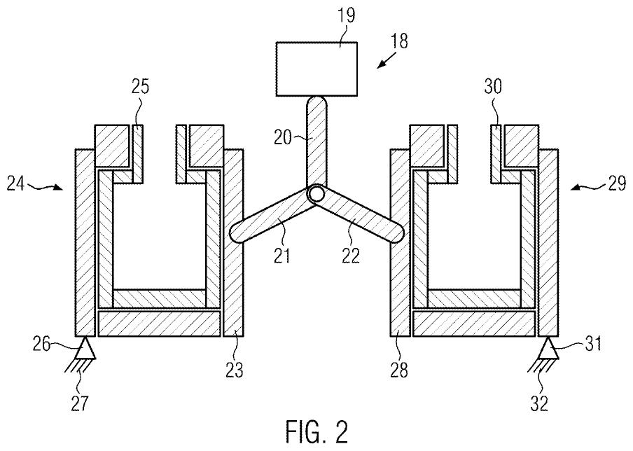

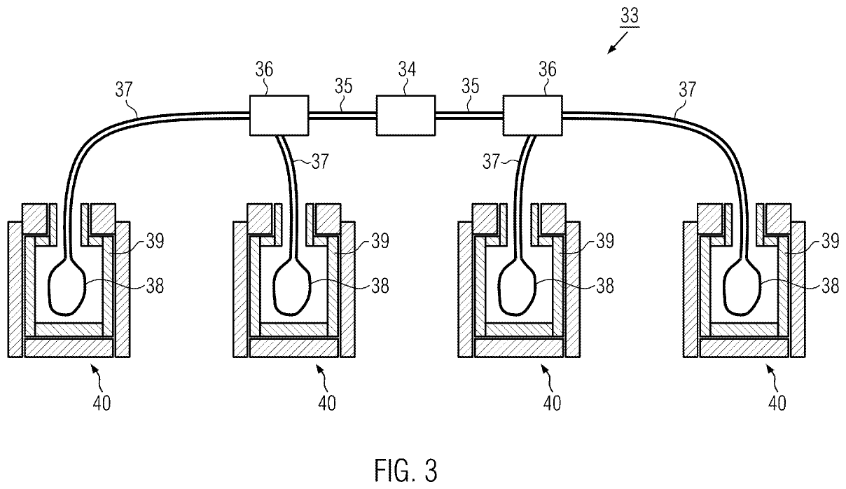

The device 1 may comprise at least one transmission device for applying pressing force to at least two of the press molds, which are not shown here but are described in FIGS. 2 and 3. Due to the applied pressing force, the fiber-containing pulp accumulated on the inner surface of the preform mold or press mold 8, 13 can be pressed. In this case, at least a part of the water which may be contained in the fiber-containing pulp may be pressed out. This part of the water can leave the preform mold or press mold 8, 13 via an outlet 10, 15.

In order to be able to collect the water exiting the outlet, a device for collecting and returning the water is provided below the stations 2, 3 in the direction of action of gravity. The device for collecting and returning the water comprises an annular trough 17 and an outer lateral surface 16 of a circular cone that is inclined toward the trough 17. The exiting water can flow along the slope of the outer lateral surface 16 to the annular trough 17.

The device may comprise at least one spray head through which cleaning medium can be sprayed into the region of the stations. The cleaning medium may, for example, be or comprise water. By way of example, a spray head is arranged just below reference sign 4 in FIG. 1.

Furthermore, the device 1, not shown here, may comprise a removal device for removing the containers comprising fibers from the preform molds or press molds, a filling device for simultaneously filling the plurality of containers comprising fibers with product, a closing device for simultaneously closing the plurality of containers comprising fibers and/or a labeling device for simultaneously or successively labeling the containers comprising fibers. Some or all of these elements may be separate rotary devices which can be connected to one another by transport stars and/or by conveyor belts and/or by a long-stator linear motor system.

FIG. 2 shows a sectional representation of a first embodiment of a transmission device 18 for applying pressing force to two press molds 24, 29. The transmission device 18 comprises a motor 19 and a three-armed lever 20, 21, 22 which can be driven by the motor 19. A pressure can thus be exerted on a first wall 23 of the one press mold 24 and on a second wall 28 of the other press mold 29. As a result, pressure can be exerted on fiber-containing pulp 25, 30 accumulated on inner surfaces of the press molds 24, 29 and a container comprising fibers can be formed in each case. Water that is present in the fiber-containing pulp can be at least partially pressed out by the pressing. This part of the water 27, 32 can leave the press molds 24, 29 through provided outlets 26, 31 and may possibly be collected for further use.

FIG. 3 shows a sectional representation of a second embodiment of a transmission device 33 for applying pressing force to four press molds 40. In the press molds 40, fiber-containing pulp 39 has accumulated on each of the inner surfaces and an elastic balloon 38, which is inflatable by means of compressed air, is located inside each press mold 40. In the inflated state, the elastic balloon 38 can press the accumulated fiber-containing pulp 39 against the inner surface of the press mold, as a result of which a container comprising fibers can be formed.

The compressed air is provided in a supply device 34, for example a pressure vessel. The supply device 34 is connected to two distributors 36 by means of two first pipes 35. One of the distributors is in each case connected to two of the elastic balloons 38 via two second pipes 37. For each of the elastic balloons 38, a valve for closing may be provided in order to allow the compressed air to be left in the elastic balloon for a given period of time after the introduction of the compressed air.

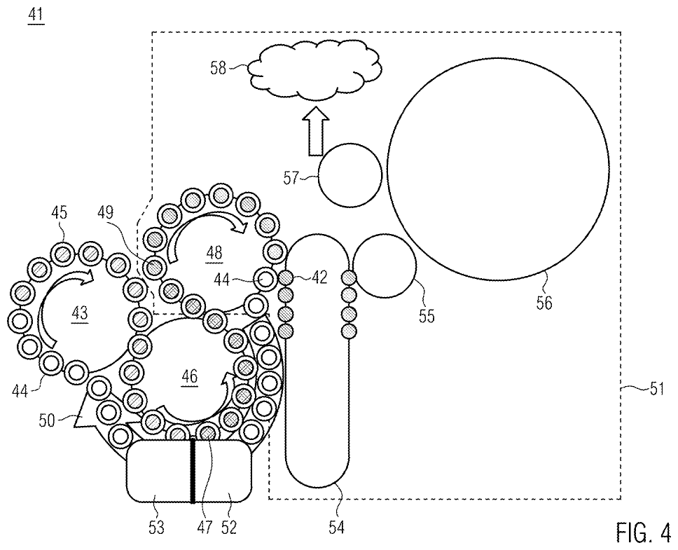

FIG. 4 shows a plan view of a device 41 for simultaneously producing a plurality of containers 42 comprising fibers, using fiber-containing pulp.

The device 41 comprises a first rotary carousel 43 and the stations and thus the preform molds and press molds are arranged on a circumference of the first carousel 43. The empty preform molds and press molds are identified by reference sign 44 and by the inner circle not being filled. The first carousel 41 is designed for the injection of the fiber-containing pulp into the preform molds and press molds. The preform molds and press molds with the injected fiber-containing pulp comprising water are identified by reference sign 45 and by a first hatching of the inner circle. The preform molds and press molds may be heated by means of an optional heating device.

The preform molds and press molds 45 with the injected fiber-containing pulp comprising water are transferred to a second rotary carousel 46, for example by means of one or more transfer stars (which may comprise spacing delay stars). The second carousel 46 is arranged downstream of the first carousel 43. The second carousel 46 is designed for internal drying of fiber-containing pulp that has been injected into the preform mold and press mold 45. Internal drying may take place by means of warm air. The preform molds and press molds, in which the injected fiber-containing pulp comprising water was dried from the inside, are identified by reference sign 47 and by a second hatching of the inner circle. Since the internal drying requires some time, the preform molds and press molds in which the injected fiber-containing pulp comprising water has already been dried from the inside can be found only after a certain circulation on the second carousel 46.

The preform molds and press molds 47, in which the injected fiber-containing pulp comprising water was dried from the inside, are transferred to a third rotary carousel 48, for example by means of one or more transfer stars (which may comprise spacing delay stars). The third carousel 48 is arranged downstream of the second carousel 46. The third carousel 48 is designed for pressing the injected fiber-containing pulp dried from the inside and for applying an inner coating. The third carousel 48 is arranged in an aseptic chamber 51. The preform molds and press molds, in which the injected fiber-containing pulp dried from the inside was pressed and an inner coating was applied, are identified by reference sign 49 and by a third hatching of the inner circle.

The device 41 furthermore comprises a return device 50 for returning to the first carousel 43 the preform molds and press molds 44 after the removal of the containers comprising fibers. The containers comprising fibers can be removed from the preform molds and press molds at a removal position of the third carousel 48. For this purpose, the preform molds and press molds can be opened.

The device 41 also comprises a cleaning device 52 and an inspection device 53 for preform molds and press molds 44 returned by means of the return device 50. Before being used again by the first carousel 43, the preform molds and press molds 44 may be cleaned and inspected. If necessary, defective preform molds and press molds 44 can be discharged.

The device 41 furthermore comprises a drying device 54 for drying, from the outside, the containers 42 comprising fibers and removed from the preform molds and press molds. The containers dried from the outside are transferred by means of a transfer star 55 to a filling device 56 and can be filled there with product. The filled containers are transferred from the filling device 56 to a closing device 58 by means of a transfer star 57 and can be closed there. The drying device 54, the filling device 56 and the closing device 58 are arranged in the aseptic chamber 51.

1. Device for simultaneously producing a plurality of containers comprising fibers, using fiber-containing pulp.

2. Device according to claim 1, comprising a plurality of stations for the respective forming of a container comprising fibers.

3. Device according to claim 2, furthermore comprising a device for feeding fiber-containing pulp, wherein the device for feeding fiber-containing pulp comprises a rotary distributor.

4. Device according to claim 1, furthermore comprising a device for collecting and returning at least a part of the water comprised in the fiber-containing pulp, wherein the device for collecting and returning at least a part of the water comprised in the fiber-containing pulp comprises the rotary distributor,
wherein the device for collecting and returning at least a part of the water comprised in the fiber-containing pulp is arranged below the plurality of stations in the direction of action of gravity,
wherein the device for collecting and returning comprises a trough an annular trough, designed as a water lock,
wherein an outer lateral surface of a circular cone inclined toward the trough is also provided below the plurality of stations in the direction of action of gravity.

5. Device according to claim 2, furthermore comprising at least one transmission device for applying pressing force to at least two of the press molds,
wherein the at least one transmission device comprises:
a motor,
at least one lever at least one toggle lever, which can be driven by the motor, whereby a compressive pressure can be exerted on the at least two of the pressing molds, and/or
wherein the at least one transmission device comprises:
a supply device for compressed air,
wherein the supply device is connected to at least two elastic balloons by means of a pipe system for distributing the compressed air, wherein each of the elastic balloons is provided for introduction into one of the press molds and for inflation by means of the compressed air.

6. Device according to claim 4, wherein the device is designed to provide a separable connection of the plurality of containers using the fiber-containing pulp during the simultaneous production of the plurality of containers comprising fibers,
wherein the separable connection comprises one or more perforations,
wherein the separable connection comprises bridges and/or a continuous planar connection between the plurality of containers.

7. Device according to claim 1, furthermore comprising a removal device for removing the containers comprising fibers from the preform molds or press molds comprising a filling device for simultaneously filling the plurality of containers with product furthermore comprising a closing device for simultaneously closing the plurality of containers furthermore comprising a labeling device for simultaneously or successively labeling the containers.

8. Device according to claim 4, furthermore comprising a device for producing the fiber-containing pulp, wherein the device for producing the fiber-containing pulp is line-connected by a feed line to the device for collecting and returning at least a part of the water comprised in the fiber-containing pulp, for feeding the at least one part of the water comprised in the fiber-containing pulp and for using the at least one part of the water comprised in the fiber-containing pulp for the production of further fiber-containing pulp,
wherein the feed line comprises a disinfection device for disinfecting the at least one part of the water comprised in the fiber-containing pulp, wherein the disinfection device is designed to dispense a disinfectant into the at least one part of the water comprised in the fiber-containing pulp and/or to apply UV-A, UV-B and/or UV-C radiation to irradiate the at least one part of the water comprised in the fiber-containing pulp.

9. Device according to claim 8, furthermore comprising a regulating device for measuring a quality of the at least one part of the water comprised in the fiber-containing pulp and for regulating the disinfection device according to a result of the measurement of the quality.

10. Device according to claim 2, wherein the device comprises a rotary carousel, and wherein the stations are provided on a circumference of the rotary carousel, wherein the rotary carousel is designed for injecting the fiber-containing pulp into the preform mold and press mold, wherein each of the preform molds and press molds is assigned an inlet valve, wherein a heating device for the preform molds and press molds is provided.

11. Device according to claim 10, wherein the device comprises a second rotary carousel arranged downstream of the rotary carousel, wherein the second rotary carousel is designed for internal drying of fiber-containing pulp injected into the preform mold and press mold, and/or
wherein the device comprises a third rotary carousel which is arranged downstream of the rotary carousel, wherein the third rotary carousel is designed for pressing fiber-containing pulp injected into the preform mold and press mold and for applying an inner coating, wherein the third rotary carousel is arranged in an aseptic chamber.

12. Device according to claim 11, wherein the rotary carousel is furthermore designed for pressing fiber-containing pulp injected into the preform mold and press mold.

13. Device according to claim 11, furthermore comprising a return device for returning to the rotary carousel the preform molds and press molds after removal of containers comprising fibers, furthermore comprising a cleaning device and an inspection device for preform molds and press molds returned by means of the return device, wherein the inspection device comprises a discharge device for defective preform molds and press molds.

14. Device according to any one of claim 11, wherein the device comprises a drying device for drying, from the outside, the containers comprising fibers and removed from the preform molds and press molds, a filling and closing device and a labeling device, wherein the drying device and the filling and closing device are arranged in an aseptic chamber.

15. Device according to claim 1, wherein the device is designed in such a way that the containers comprising fibers can be arranged in such a way that a longitudinal axis of the containers encloses an angle between 70° to 110° to a direction of action of gravity.

16. Device according to claim 1, wherein the device has a modular design.

17. Method for simultaneously producing a plurality of containers comprising fibers, using fiber-containing pulp by means of the device according to claim 1.

18. Device according to claim 2 wherein each station comprises a preform mold and a press mold, wherein the preform mold comprises a lattice structure.
